(19) United States
(12) Patent Application Publication
(10) Pub. No.: US 2022/0117785 A1
(43) Pub. Date: Apr. 21, 2022

(54) DEVICE FOR MOUNTING AND DEMOUNTING A HOLLOW NEEDLE ON AND FROM A PHACO INSTRUMENT HANDPIECE

(71) Applicant: WEFIS GmbH, Köln (DE)

(72) Inventor: Dietrich WERNZ, Köln (DE)

(21) Appl. No.: 17/603,077

(22) PCT Filed: Feb. 19, 2020
(86) PCT No.: PCT/EP2020/054376

§ 371 Date: Oct. 12, 2021

(30) Foreign Application Priority Data
Apr. 26, 2019 (EP) 19171418.7

(51) Int. Cl. A61F 9/007

(52) U.S. Cl. CPC A61F 9/00763; A61B 2090/031; A61F 9/00745

(57) ABSTRACT

The device (10) for mounting and demounting a hollow needle (12) on or from a phaco instrument handpiece (14) comprises a housing (16) having a torque limiting and non-return coupling (34). The coupling (34) is provided with corresponding coupling release projections (58) and coupling recesses (56) on two coupling surfaces (36), (38). Said coupling surfaces (36, 38) further have corresponding non-return projections (62) and non-return recesses (60). The coupling (34) is formed by a driver element (32) and a rotary shaft (18) having a plug-on end (22) for plugging on the hollow needle (12) to be mounted and demounted, respectively.

The invention relates to a device for mounting a hollow needle on a phaco instrument handpiece and for demounting the hollow needle from the phaco instrument handpiece. This surgical instrument may be, for example, a phacoemulsification handpiece, a phaco irrigation handpiece, a phaco suction handpiece, or a combined phaco irrigation/suction handpiece.

In ophthalmology, a surgical instrument is used to treat the eye lens, said surgical instrument having a phacoemulsification handpiece with a hollow needle screwed thereto. It is known to screw the hollow needle onto the handpiece by using torque-limiting manually operable tools. As an example of this related art, reference is made to CN-A-108992744, WO-A-2011/149727, US-A-2009/157009 and US-A-2014/0100515. The torque-limiting tools described in these two documents are quite elaborate in design.

An object of the invention is to provide a device for mounting and demounting a hollow needle on and from a phaco instrument handpiece, the device having a simple design and no torque limitation for demounting the hollow needle from the phaco instrument handpiece.

To achieve this object the invention provides a device for mounting and demounting a hollow needle on and from a phaco instrument handpiece, the device being provided with
a manually operable housing
a rotary shaft rotatably mounted in the housing, the rotary shaft comprising a plug-on end extending outwards from an opening of the housing for plugging on the hollow needle, the plug-on end comprising internal engaging projections for engaging with recesses in the outside of the hollow needle for the purpose of rotating the hollow needle when manually rotating the housing,
a torque limiting and non-return coupling having a contact surface arranged at the rotary shaft as a first coupling surface, and
a driver element arranged coaxially to the rotary shaft in the housing, being coupled to the housing, and having a radial contact surface as a second coupling surface of the torque limiting and non-return coupling,
wherein the two coupling surfaces rest against each other by being spring-loaded against each other,
wherein the one coupling surface has at least one coupling release projection and the other coupling surface has at least one coupling recess being complementary to the form of the coupling release projection,
wherein the one coupling surface has at least one non-return projection and the other coupling surface has at least one non-return recess,
wherein the at least one non-return recess—viewed in circumferential direction of a circular line concentrically to the axis of the rotary shaft—has an extension being larger, in particular more than twice larger than the extension of the non-return projection in circumferential direction of the circular line,
wherein the at least one non-return projection rests against one of the two ends of the non-return recess arranged as viewed in the circumferential direction of the circular line when the at least one coupling release projection is immersed in the at least one coupling recess,
wherein the at least one non-return projection—viewed in axial extension of the rotary shaft—has a height which is smaller than the height of the at least one coupling release projection, and
wherein the at least one non-return projection is entirely moved out of the at least one non-return recess when the at least one coupling release projection is moved out of the at least one coupling recess, and moved above or on the coupling surface comprising the non-return recess, until the at least one coupling release projection is again immersed in the at least one coupling recess when the housing continues to rotate.

Thus, the invention provides a device having a manually operable housing which can be gripped by the fingers of a hand and in which a rotary shaft is rotatably mounted. Furthermore, a driver element is arranged inside the housing, wherein the driver element can be part of the housing or can be an element separate from the housing. The driver element rotates along with the housing when the housing is rotated by hand. Between the driver element and the rotary shaft there is a torque limiting and non-return coupling which has two coupling surfaces resiliently pressed against each other. One of said coupling surfaces is located on the rotary shaft, while the other coupling surface is located on the driver element. Typically, both coupling surfaces are configured as radial contact surfaces extending radially to the rotary shaft.

The rotary shaft also comprises a plug-on end which is led out from an opening of the housing and forms a receiving recess that is plugged on the hollow needle when the hollow needle is to be mounted or demounted. The hollow needle is typically provided with recesses on its outside into which engaging projections on the inside of the receiving recess of the plug-on end immerse. Alternatively, the hollow needle can have engaging projections on its outside to be received in recesses on the inside of the receiving recess of the plug-on end. In both cases, a kind of form-fit is created, whereby the hollow needle is also rotated when the housing is rotated manually.

The two coupling surfaces are provided with coupling release projections and coupling recesses that are complementary with respect to their form. The projections can all be arranged on the one coupling surface and the complementary coupling recesses can all be arranged on the other coupling surface. However, it is also possible that projections and recesses are formed on both coupling surfaces. According to the invention, at least one coupling release projection and at least one coupling recess are required. Due to the resilient pretensioning of the two coupling surfaces, said coupling surfaces rest against each other and the coupling release projections are immersed in their associated coupling recesses until the torque exceeds a limit value. The size of this value depends on design, for example, one the one hand, on the force with which the two coupling surfaces rest against each other, and, on the other hand, on the design (i.e. geometry) of the coupling release projections and coupling recesses. Typically, the coupling release projections and the coupling recesses are spherically formed (for example as a hemisphere or half shall).

To ensure that the torque limitation is only effective when the hollow needle is screwed onto the phaco instrument handpiece (hereafter referred to as a phacoemulsification handpiece, representative of such instruments), a non-return device is provided in the device according to the invention. Said non-return device comprises non-return projections and non-return recesses at the coupling surfaces, wherein it also applies here that all non-return projections are arranged on the one coupling surface and all non-return recesses are arranged on the other coupling surface, or that both coupling surfaces have non-return projections and non-return recesses. According to the invention, at least one non-return projections and at least one non-return recess are required.

The relative arrangement of the non-return projections and the non-return recessed is selected according to the invention such that each non-return projection can shift in its associated non-return recess in the one direction of rotation of the housing about the axis of the rotary shaft, this direction of rotation being the direction in which the torque limitation acts. In this direction of rotation, the coupling release projections then move out of their coupling recesses when the maximum permissible torque is reached, spacing both coupling surfaces by the height of the coupling release projections. In this condition, the non-return projections are also moved out of their non-return recesses. According to the invention, the non-return projections are now flatter than the coupling release projections, i.e. they have a lower height than the coupling release projections when viewed in the axial extension of the rotary shaft. Each non-return recess has two ends or end stops located in the direction of rotation of the housing and opposite thereto. Before a non-return projection, which is immersed in the non-return recess associated thereto, reaches the end located in the direction of rotation in which the torque limitation acts, the non-return projection is already lifted out of the non-return recess when the torque limitation is exceeded, since the coupling release projections are moved out of the coupling recesses. In contrast, each non-return projection in the non-return recess already rests against its other end when the coupling release projections are positioned in the coupling recesses. This means that the non-return projections in the opposite direction of rotation do not allow the coupling release projections to move out of the coupling recesses, which means that the torque limitation in this opposite direction of rotation is cancelled and therefore inactive.

The coupling release projections and the coupling recesses can be arranged on circular lines that are concentric to the axis of the rotary shaft and are aligned with each other, i.e. have the same radius. Similarly, the non-return projections and the non-return recesses may be arranged on circular lines that are aligned with each other. Typically, however, the circular lines of the coupling release projections and recesses are different from those of the non-return projections and recesses, but this does not necessarily be the case.

In a further expedient embodiment of the invention, it may be provided that the one coupling surface has several coupling release projections and the other coupling surface has a number of coupling recesses equal to the number of coupling release projections, or has a number of coupling recesses which is larger by a factor of 2 or 3 or more than the number of coupling release projections, wherein the relative arrangement of a number of coupling recesses equal to the number of coupling release projections is equal to the relative arrangement of the coupling release projections.

In a further expedient embodiment of the invention, it may be provided that the one coupling surface has a larger number of non-return recesses than there are non-return projections on the other coupling surface.

It may also be expedient if the number of non-return recesses is equal to or larger than the number of non-return projections by a factor of 2 or 3 or more.

It may also be advantageous if the one coupling surface has three coupling release projections each offset by 1200 relative to one another and the other coupling surface has six coupling recesses each offset by 60° relative to one another, and if the one coupling surface has three non-return projections each offset by 1200 relative to one another and the other coupling surface has six non-return recesses each offset by 600 relative to one another, wherein the non-return recesses are each arranged in alignment with the center between two coupling recesses.

In a further expedient embodiment of the invention, it may be provided that the at least one or each coupling release projection and the at least one or each coupling release recess are arranged on a first circular line being concentric to the rotary shaft, and that the at least one or each non-return projection and the at least one or each non-return recess are arranged on a second circular line being concentric to the rotary shaft and different from the first circular line. However, it is also possible that coupling release recesses and coupling release projections are distributed along several first circular lines and that correspondingly non-return projections and non-return recesses are distributed along several second circular lines.

In a further expedient embodiment of the invention, it is provided that the rotary shaft in the housing is rotatably mounted by means of bearing bushes, and/or that the rotary shaft extends through the driver element, and/or that a helical compression spring is arranged concentrically on the rotary shaft, the helical compression spring extending and being supported between a support surface in the hosing and a support surface on the rotary shaft, and/or that the driver element is pressed by a helical compression spring against the rotary shaft.

Finally, it is also conceivable that the at least one or each engaging projection and/or the at least one or each coupling release projection is spherically formed, and/or that the at least one or each non-return projection and the at least one or each non-return recess comprise contact surfaces extending at right angles or substantially at right angles to the coupling surfaces and resting against each other when the at least one coupling release projection is immersed in the at least one coupling recess.

The device according to the invention is expediently made entirely or quite predominantly of plastic material (for example in the form of plastic injection-molded parts), which applies in particular to the coupling. The more non-return projections are provided, the more shear-resistant the coupling surfaces rest against each other when it comes to unscrewing the hollow needle.

In the following, the invention is described in more detail by means of an exemplary embodiment and with reference to the drawing, in which

FIG. 1 is a perspective view of an exemplary embodiment of the device for mounting and demounting a hollow needle on and from a phacoemulsification handpiece,

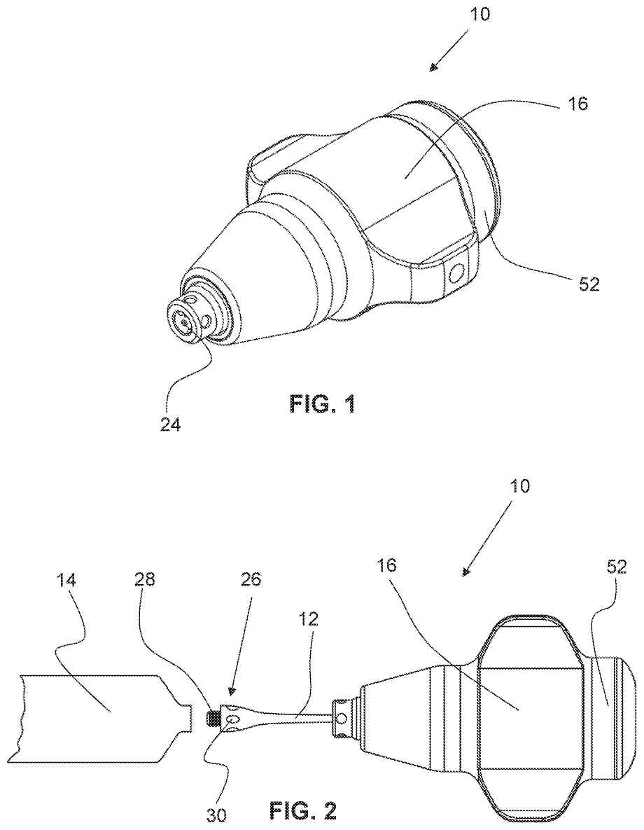

FIG. 2 is a side view of the device of FIG. 1 indicating its sliding onto a hollow needle for mounting on a phacoemulsification handpiece,

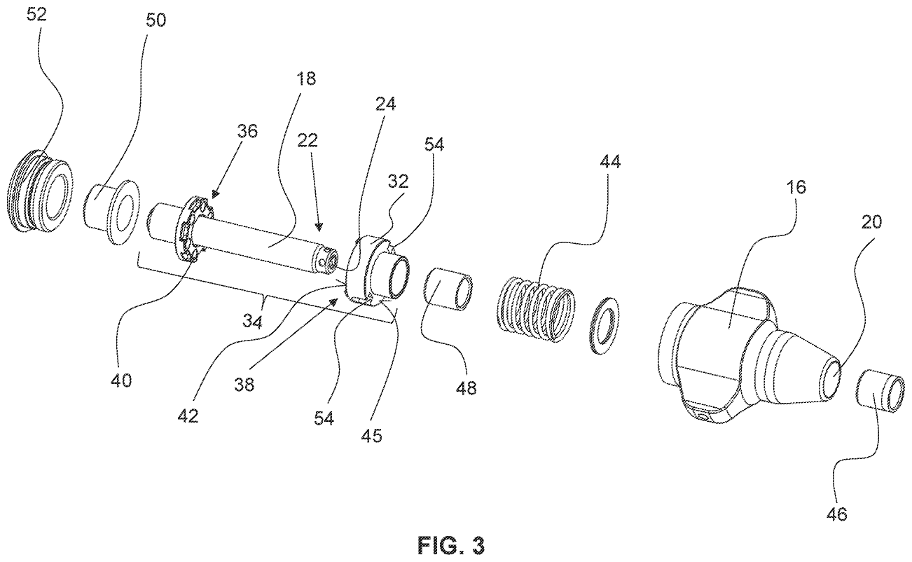

FIG. 3 is a perspective and exploded view of the structure of the device of FIG. 1,

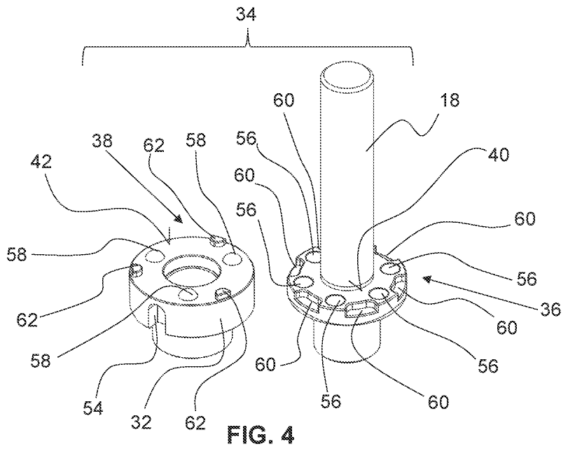

FIG. 4 is a perspective view of the coupling surfaces of the torque limiting and non-return coupling of the device of FIG. 1,

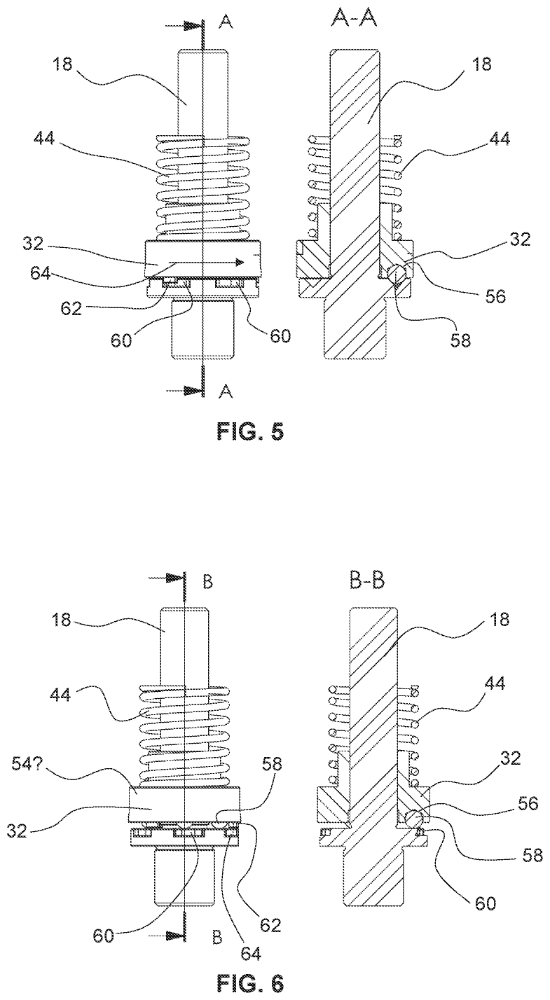

FIGS. 5 and 6 are side views and cross-sectional views of the coupling in two different twisting positions of the two coupling parts,

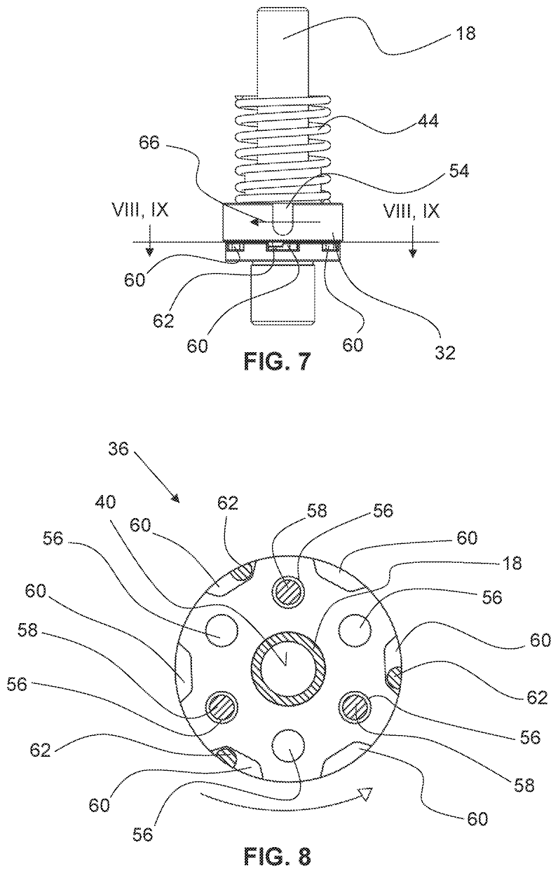

FIG. 7 is a side view of the coupling with its coupling surfaces engaged, and

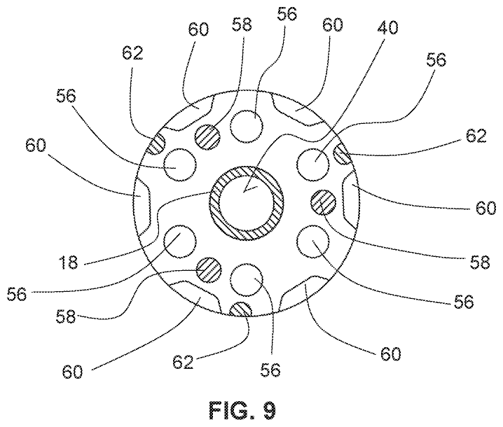

FIGS. 8 and 9 are sectional views corresponding to VIII-IX of FIG. 7.

FIGS. 1 to 3 are different views of an exemplary embodiment of a device 10 for mounting a hollow needle 12 on a phacoemulsification handpiece 14 and for demounting the hollow needle 12 from the phacoemulsification handpiece 14. The device 10 comprises a housing 16 which can be gripped by the fingers of a hand and in which a rotary shaft 18 is rotatably mounted, the rotary shaft 18 extending outwards through an opening 20 of the housing 16. This end of the rotary shaft 18 is the plug-on end 22 which is hollow and has internal engaging projections 24 indicated, inter alia, in FIGS. 1 and 2. The hollow needle 12 has a conical portion 26 with a threaded socket 28 adjoining thereto. Within the conical portion 26, the hollow needle 12 has recesses 30 on its outside into which the engaging projections 24 engage when the hollow needle 12 is entirely located within the housing 16.

The rotary shaft 18, together with a driver element 32, forms a torque limiting and non-return coupling 34 (hereinafter referred to as a coupling for convenience only) having a first coupling surface 36 and a second coupling surface 38. The first coupling surface 36 is formed in this embodiment as a radial contact surface 40 on the rotary shaft 18, while the second coupling surface 38 is formed as a radial contact surface 42 of the driver element 32. Both coupling surfaces 36, 38 are resiliently tensioned against each other by means of a spring 44, which in this exemplary embodiment is designed as a spiral compression spring. The rotary shaft 18 is rotatably mounted within the housing 16 via bearing sleeves 46, 48, 50. Meanwhile, the rotary shaft 18 penetrates the driver element 32. The spring 44, which is seated coaxially on the rotary shaft 18, is located on the side of the driver element 32 facing away from the first coupling surface 36. A closing cover 52 is located at the end of the housing 16 facing away from the opening 20 for the plug-on end 22 of the rotary shaft 18. The spring 44 is supported, on the one hand, on a support surface in the housing 16 (not shown) and, on the other hand, on a support surface 45 arranged on the driver element 32, facing away from the second coupling surface 38 of the driver element 32.

The driver element 32 is arranged in the housing 16 in a form-fit manner. For this purpose, the driver element 32 has two circumferential recesses 54 which engage with internal driver lugs (not shown) in the housing 16. Thus, when the housing 16 is rotated, the driver element 32 is rotated therein as well, wherein it can shift axially on the rotary shaft 18 without that the driver lugs of the housing 16 become disengaged from the circumferential recesses 54.

The essential element of the device 10 is the coupling 34, which is shown again in perspective view in FIG. 4. The coupling 34 is ultimately formed by the rotary shaft 18 and the driver element 32. In this exemplary embodiment, the first coupling surface 36 has six coupling recesses 56 that selectively cooperate with three coupling projections 58 of the second coupling surface 38. In this exemplary embodiment, the first coupling surface 36 further includes six non-return recesses 60 that cooperate with three non-return projections 62 of the second coupling surface 36.

The coupling 34 according to the invention serves to provide a torque limitation in the one of two opposite rotational movements of the housing 16. This rotational movement is the rotational movement by which the housing must be rotated in order to screw on the hollow needle 12. The further function of the coupling 34 is to exclude torque limitation in the opposite direction of rotation of the housing 16. In the following, this non-given torque limitation is also called “non-return”, which means that the torque limitation is blocked, i.e. cancelled.

FIG. 5 shows the situation in which the three coupling release projections 58 are immersed in three of six coupling recesses 56. In this condition, the three non-return projections 62 are also immersed in three of the six non-return recesses 60. The driver element 32 is arranged in the housing 16 in a torque-proof manner. If the housing 16 is now rotated so that the driver element 32 rotates along in the direction of the arrow 64 (see FIG. 5), the driver element 32 drives the rotary shaft 18 as long as the torque given by the strength of the spring 44 and by the contact pressure of the two coupling surfaces as well as by the design of the coupling release projections 58 and the coupling recesses 56 is not exceeded. Once this occurs, the coupling release projections 58 move out of the coupling recesses 56, causing the driver element 32 to shift axially within the housing 16 against the force of the spring 44, as shown in FIG. 6. In order for this to function in said screw-on rotational movement of the housing 16, the non-return projections 62 must also be able to move out of the non-return recesses 60. This in turn is achieved by the fact that the non-return recesses 60 have a greater extension than the non-return projections 62 when viewed in the circumferential direction of the rotary shaft 18. This is shown, for example, in FIG. 5. The non-return recesses 60 are approximately twice as wide (viewed in the circumferential direction of the rotary shaft 18) as the non-return projections 62.

In the condition of the coupling engagement (see, e.g. FIG. 5), the non-return projections 62 rest on the one end of the non-return recesses 60 and are free to the other end of the non-return recesses 60. This free distance is now sufficient to allow the driver element 32 to continue to rotate relative to the rotary shaft 18 when the torque limit is reached, allowing the coupling release projections 58 to move out of the coupling release recesses 56, with the result, that the non-return projections 62 are now above the first coupling surface 36, and thus the driver element 32 can continue to rotate until the coupling release projections 58 re-immerse in the coupling release recesses 56 closest in the direction of rotation. Then, the non-return projections 62 also re-immerse in the nearest non-return recesses 60. This process of “coupling slippage” then continues as the housing 16 is further rotated, signaling to the user (possibly also by appropriate acoustics) that the maximum permissible torque for screwing the hollow needle 12 onto the phacoemulsification handpiece 14 has been reached.

In the reverse case, i.e. when the housing 16 is twisted to unscrew the hollow needle 12, the initial situation is again as shown in FIG. 5 and FIG. 7, respectively. If the housing 16 is now twisted in such a way that the driver element 32 rotates in the direction of the arrow 66, the non-return projections 62 block a relative twisting of the driver element 32 and the rotary shaft 18, since, viewed in the direction of rotation of the arrow 66, they rest directly against the edges of the non-return recesses 60 located in this direction of rotation 66.

FIGS. 8 and 9 illustrate the functions described above. The starting point is FIG. 8, which shows coupling 34 in engagement. The three coupling release projections 58 are located in three of the six coupling recesses 56, while the three non-return projections 62 are immersed in three of the six non-return recesses 60, specifically, with reference to FIG. 8, at their boundaries as viewed in the clockwise direction. Now, as shown in FIG. 9, when the housing 16 is rotated to screw on the hollow needle 12 (see the arrow 64), the coupling release projections 58 move out of the coupling recesses 56 when the maximum torque is exceeded. Since the non-return projections 62 have a lower height than the coupling release projections 58 when viewed in the axial direction, the non-return projections 62 move above the relevant coupling surface 36, thus allowing the torque limitation. In the reverse direction of rotation, i.e. opposite to the arrow 64 of FIG. 9, the non-return projections 62 block the torque limitation and thus cancel it. The driver element 32 will therefore drive the rotary shaft 18 in any case, which is advantageous for unscrewing the hollow needle 12 from the phacoemulsification handpiece 14.

### LIST OF REFERENCE NUMERALS

10 device
12 hollow needle
14 phacoemulsification handpiece
16 housing
18 rotary shaft of housing
20 opening of housing
22 plug-on end of rotary shaft
24 engaging projections
26 conical portion of hollow needle
28 threaded socket of hollow needle
30 recesses in portion 26
32 driver element of housing
34 torque limiting and non-return coupling
36 first coupling surface
38 second coupling surface
40 first contact surface
42 second contact surface
44 spring
45 support surface on driver element for the spring
46 bearing sleeve
48 bearing sleeve
50 bearing sleeve
52 closing cover of housing
54 circumferential recesses on driver element
56 coupling recesses
58 coupling release projection
60 non-return recesses
62 non-return projections
64 arrow (direction of rotation)
66 arrow (direction of rotation)

1. A device for mounting and demounting a hollow needle on and from a phaco instrument handpiece, comprising
a manually operable housing,
a rotary shaft rotatably mounted in the housing, the rotary shaft comprising a plug-on end extending outwards from an opening of the housing for plugging on the hollow needle, the plug-on end comprising internal engaging projections for engaging with recesses in the outside of the hollow needle for the purpose of rotating the hollow needle when manually rotating the housing,
a torque limiting and non-return coupling having a contact surface arranged at the rotary shaft as a first coupling surface, and
a driver element arranged coaxially to the rotary shaft in the housing, being coupled to the housing and having a contact surface as a second coupling surface of the torque limiting and non-return coupling,
wherein the two coupling surfaces rest against each other by being spring-loaded against each other,
wherein the one coupling surface has at least one coupling release projection and the other coupling surface has at least one coupling recess being complementary to the form of the coupling release projection,
wherein the one coupling surface has at least one non-return projection and the other coupling surface has at least one non-return recess,
wherein the at least one non-return recess—viewed in circumferential direction of a circular line concentrically to the axis of the rotary shaft—has an extension being larger, in particular more than twice larger than the extension of the non-return projection in circumferential direction of the circular line,
wherein the at least one non-return projection rests against one of the two ends of the non-return recess arranged as viewed in the circumferential direction of the circular line when the at least one coupling release projection is immersed in the at least one coupling recess,
wherein the at least one non-return projection, when viewed in axial extension of the rotary shaft, has a height which is smaller than the height of the at least one coupling release projection, and
wherein the at least one non-return projection is entirely moved out of the at least one non-return recess when the at least one coupling release projection is moved out of the at least one coupling recess, and moved above or on the coupling surface comprising the non-return recess, until the at least one coupling release projection is again immersed in the at least one coupling recess when the housing continues to rotate.

2. The device according to claim 1, wherein the one coupling surface comprises a plurality of coupling release projections and the other coupling surface comprises a number of coupling recesses equal to the number of coupling release projections or a number of coupling recesses larger by factor 2 or 3 than the number of coupling release projections, wherein the relative arrangement of a number of coupling recesses equal to the number of coupling release projections is equal to the relative arrangement of the coupling release projections.

3. The device according to claim 1, wherein the coupling surface has a larger number of non-return recesses than non-return projections are arranged at the other coupling surface.

4. The device according to claim 2, wherein the number of non-return recesses is equal to or larger by factor 2 or 3 or more than the number of non-return projections.

5. The device according to claim 1, wherein the one coupling surface has three coupling release projections each arranged offset by 120° relative to one another, and the other coupling surface has six coupling recesses each arranged offset by 60° relative to one another, and that the one coupling surface has three non-return projections arranged offset by 120° relative to one another, and the other coupling surface has six non-return recesses arranged offset by 60° relative to one another, wherein the non-return recesses are each arranged in alignment with the center between two coupling recesses.

6. The device according to claim 1, wherein the at least one or each coupling release projection and the at least one or each coupling release recess are arranged on a first circular line being concentric to the rotary shaft, and that the at least one or each non-return projection and the at least one or each non-return recess is arranged on a second circular line being concentric to the rotary shaft and being different from the first circular line.

7. The device according to claim 1, wherein the rotary shaft in the housing is rotatably mounted by means of bearing bushes and/or that the rotary shaft extends through the driver element and/or that a helical compression spring is arranged concentrically on the rotary shaft, the helical compression spring extending and being supported between a support surface in the hosing and a support surface on the rotary shaft.

8. The device according to claim 1, wherein the at least one or each engaging projection and the at least one or each coupling release projection are spherically formed, and that the at least one or each non-return projection and the at least one or each non-return recess comprise contact surfaces extending at right angles or substantially at right angles to the coupling surfaces and abutting each other when the at least one coupling release projection is immersed in the at least one coupling recess.

9. The device according to claim 2, wherein the coupling surface has a larger number of non-return recesses than non-return projections are arranged at the other coupling surface.

10. The device according to claim 3, wherein the number of non-return recesses is equal to or larger by factor 2 or 3 or more than the number of non-return projections.
